(19) United States
(12) Patent Application Publication
(10) Pub. No.: US 2011/0274795 A1
(43) Pub. Date: Nov. 10, 2011

(54) IN-LINE DEAERATION PROCESS FOR THE PRODUCTION OF SELF-SUPPORTING FILM PRODUCTS

(75) Inventors: Beuford A. BOGUE, New Carlisle, IN (US); Garry L. MYERS, Kingsport, IN (US); Patrick DAILEY, Merrillville, IN (US); Greg SLOMINSKI, Valparaiso, IN (US)

(73) Assignee: MONOSOL RX, LLC, Warren, NJ (US)

(21) Appl. No.: 12/774,292

(22) Filed: May 5, 2010

(51) Int. Cl. G01N 33/02; A23P 1/00

(52) U.S. Cl. 426/231; 426/486; 99/450.1

(57) ABSTRACT

The present invention provides processes and systems for preparing self-supporting film-forming compositions. The process generally includes the steps of: (a) providing a self-supporting film-forming matrix including an edible polymer component and a fluid carrier selected from the group consisting of water organic solvents and combinations thereof: (b) degassing the matrix via the steps of: (i) directing a flow of the matrix having a first density through at least one volume reduction device: and (ii) increasing the density of the matrix to form a second density. The present invention further includes the addition of actives to the matrix so as to provide an active-containing degassed film forming matrix.

### FIELD OF THE INVENTION

The present invention relates to the process for producing self-supporting consumable film with desirable properties and characteristics. Specifically, the present invention relates to processes and systems for producing self-supporting film or sheet products, such as thin film, multi-layered film, wafers and the like, including advantageous deaeration processing of the mixture(s) of components to be formed into the final product sheets and/or dosages.

### BACKGROUND OF RELATED TECHNOLOGY

When films are manufactured, and particularly ingestible active-containing films, the components of the film-forming mixture from which the final product(s) are formed may be expensive, volatile, degradable, highly reactive, and combinations thereof. Thus, it is highly desirable, yet very difficult, to efficiently and effectively form film with certain advantageous characteristics. Current processes and manufacturing designs may form film less effectively or with less predicable characteristics. Thus, there exists a current need in the art for effective methods and processes for making self-supporting consumable film products with certain characteristics.

### SUMMARY OF THE INVENTION

In one embodiment of the present invention, there is provided a process for preparing a degassed film-forming matrix including the steps of: (a) providing a film-forming pre-mix including an edible polymer component and a fluid carrier selected from the group consisting of water, organic solvents, and combinations thereof (b) mixing the pre-mix with an active component to form a film-forming matrix; and (c) degassing the film-forming matrix to provide a degassed matrix having an increased density, the degassing step including directing a flow of the film forming matrix having a first density through at least one volume reduction device, where the flow through the volume reduction device increases the density of the matrix to form a matrix having a second density, the second density being a higher density than the first density.

In another embodiment of the present invention, there is provided a process for preparing a self-supporting film-forming composition including: (a) providing a film-forming matrix including an edible polymer component and a fluid carrier selected from the group consisting of water, organic solvents, and combinations thereof: (b) degassing the matrix, including the steps of: (i) directing a flow of the matrix having a first density through at least one volume reduction device; and (ii) increasing the density of the matrix to yield a flow of a resultant degassed matrix; and (c) mixing the resultant degassed matrix with an active component to form a degassed matrix.

In still another embodiment of the present invention, there is provided a process for preparing a self-supporting film-forming composition including: (a) providing a film-forming matrix including an edible polymer component and a fluid carrier selected from the group consisting of water, organic solvents, and combinations thereof; (b) degassing the film-forming matrix by directing a flow of the film-forming matrix having a first density through at least one volume reduction device, the device including a plurality of porous deaerating channels, so as to form a film-forming matrix having a second density, the second density being higher than the first density; and (c) mixing the film-forming matrix having a second density with an active component to form an active-containing degassed film-forming matrix.

In other embodiments of the present invention, there may be provided a process for preparing a self-supporting film-forming composition including: (a) forming a matrix including an edible polymer component and; a fluid carrier; (b) degassing the matrix including the steps of: directing a flow of the matrix having a first density through at least one volume reduction device, the volume reduction device including a rotatable surface, so as to form a film-forming matrix having a second density, the second density being a higher density than the first density; and (c) mixing the resultant film-forming matrix having a second density with an active component to form an active-containing degassed film-forming matrix.

In another embodiment of the present invention, there is provided a system for forming edible film, including: (a) a first mixer for combining at least one self-supporting film forming matrix, including an edible polymer component and at least one fluid carrier; (b) at least one volume reduction device for degassing the matrix; and (c) at least one second mixer to combine a quantity of a degassed matrix with a quantity of an active component to form an active-containing degassed matrix having a uniform distribution of the active therein.

The present invention with its various embodiments may be better understood through a study of the following figures and description.

### BRIEF DESCRIPTION OF THE DRAWINGS

FIG. 1 is a schematic view of an apparatus suitable for preparation of a pre-mix, addition of an active, and subsequent formation of the film.

FIG. 2 is a schematic view of an apparatus suitable for drying the films of the present invention.

FIG. 3 is a schematic view of an exemplary drying step for the film sheet of the present invention.

FIG. 4 is a perspective view of a volume reduction device used in the process and system of the present invention.

FIG. 5A is a schematic view of an alternate volume reduction device useful in the process and system of the present invention.

FIG. 5B is a cross-sectional view of the degassing device of FIG. 5A.

FIGS. 6A-B are photomicrographs showing before and after degassing (deaeration) of a composition using one embodiment of the present invention.

FIGS. 6C-D are photomicrographs showing before and after degassing (deaeration) of a composition using a second embodiment of the present invention.

### DETAILED DESCRIPTION OF THE INVENTION

The present invention is directed to processes and systems for the production of self-supporting film-forming compositions with desirable and advantageous properties. In particular, the present invention is directed to methods of deaerating a film-forming matrix prior to the formation of a final, self-supporting film product. As used herein, the terms “deaerating”, “degassing”, and “debubbling” are used interchangeably, and refer to the process of reducing or altogether removing gas bubbles in the film-forming matrix.

As used herein, the term “volume reduction device” includes any device that is capable of sufficiently reducing the volume of the film-forming matrix, such as by removing or reducing the amount of gas bubbles from a film forming matrix. Thus, a volume reduction device may, in some embodiments, be a deaerating (or degassing) device. Reduction (or elimination) of gas bubbles in a film forming matrix thus reduces the volume occupied by the matrix and increasing its measurable density. Thus, a deaerated (or degassed/debubbled) film-forming matrix will have an increased density as compared to the same film-forming matrix that has not been deaerated. It will be noted, of course, that a volume reduction device may not completely eliminate all gas bubbles from the film forming matrix, but rather reduces the amount of gas bubbles to a sufficient level.

By deaerating the film-forming compositions with the processes and systems of the present invention, the uniformity of the self-supporting film products is more maintainable, and thus the amount of active component(s) dispersed therein is predictable and able to be optimized. Thus, resulting film products formed from such improved film-forming matrix have superior properties, and the processes of the present invention may result in a higher percentage of uniform and usable film products, limiting the amount of wasted film scrap.

Film systems embody a field of technology that has major advantages in areas of administering drug, medicament, biologicals, bioeffecting agents, diagnostic reagents and various other active and agent delivery systems to an individual in need thereof. In order to provide a desirable final product which exhibits advantageous characteristics and desirable properties, the processing and manufacturing of film strips and film technology is technologically demanding and cumbersome. Any desired active component may be used, including, but not limited to those active components set forth in Applicant's co-pending patent application, U.S. Ser. No. 12/711,883, filed Feb. 24, 2010, and entitled “Use of Dams to Improve Yield In Film Processing.”

Some constituents of the film strip, including the actives set forth above, are very expensive and may be easily degraded. Thus, various constituents may be sparingly used, particularly if they are not recoverable and/or reusable. Wasted constituent in the manufacturing process results in direct loss of profitability and efficiency. As such, it is desirable to limit any waste in the manufacturing process in order to conserve costs and promote efficiency in production. One way to minimize cost is to limit the amount of wasted film composition. Some waste may occur along the way from the formation and processing of the film into the final individual-sized delivery modules or doses, while other scrap may be due, for example, to malfunctioning packaging equipment.

To appreciate the present invention, it is helpful to understand the general characteristics of individual film strip doses, the processing and manufacturing of the film strips, as well as the factors and variables which may be related to the methods and systems of the present invention. It is known and appreciated by the present inventors that additional characteristics of film strips and methods of making the same are possible and foreseeable in combination with desirable properties and characteristics listed herein, as may be desired. Thus, the present disclosure, by way of example, in no way limits the various embodiments of the present invention.

It will be understood that the term “film” includes delivery systems of any thickness, including films, sheets, discs, wafers, and the like, in any shape, including rectangular, square, or other desired shape. The film may be in the form of a continuous roll of film or may be sized to a desired length and width. The films described herein may be any desired thickness and size suitable for the intended use. For example, a film of the present invention may be sized such that it may be placed into the oral cavity of the user. Other films may be sized for application to the skin of the user, i.e., a topical use. For example, some films may have a relatively thin thickness of from about 0.1 to about 10 mils, while others may have a somewhat thicker thickness of from about 10 to about 30 mils. For some films, especially those intended for topical use, the thickness may be even larger, i.e., greater than about 30 mils. It will be understood, of course, that the thickness of the film may be limited due to the formulation used, and thicker films may require longer drying times. Further, thicker films may desirably be formed through lamination of thinner films. In addition, the term “film” includes single-layer compositions as well as multi-layer compositions, such as laminated films, coatings on films and the like. The composition in its dried film form maintains a uniform distribution of components through the application of controlled drying of the film. Films may include a pouch or region of drug between two films.

The drug may be dispersed throughout the film, or it may be deposited onto one or more surfaces of the film. In either way, the amount of drug per unit area is desirably uniform throughout the film. It is desired that the films of the present invention include a uniformity of component distribution throughout the volume of a given film. Such uniformity includes a substantially uniform amount of drug per unit volume of the film, whether the drug is within the matrix of the film or coated, laminated, or stabilized on one or more surfaces thereof. When such films are cut into individual units, the amount of the agent in the unit can be known with a great deal of accuracy.

Uniformity of drug throughout the film is important in administering an accurate and effective dose of drug to a user. Various methods of forming uniform films, as well as various additives and fillers, may be used, including those methods and materials described in U.S. Pat. Nos. 7,425,292, 7,357,891, and 7,666,337, which are herein incorporated by reference in their entireties.

Each individual film strip dose may be characterized in that it may have a piece size or strip weight, width, length, and thickness. These parameters may be varied in order to yield a dosage that dissolves, for example, quickly, slowly, over a period of predetermined length, and combinations thereof. Further, the size and compositional make-up of the dosage may attribute different levels or amounts of active component(s) or agent(s) which may be delivered to an individual. Thus, various film strip shapes and varying thicknesses are included in the film strip dosages of the present invention. In order to manufacture a film strip which meets the rigors for commercialization and regulatory approval, factors including consistency, quality, and efficacy must be maintained throughout processing and manufacture.

### Forming the Film

When the matrix, including the film-forming polymer, polar solvent (such as water, for example), any additives, and the active component is formed, this may be done in a number of steps. For example, the components may all be initially added together or pre-mixes of different materials may be prepared. The advantage of a pre-mix is that all components, except for those that may be degradable, such as the active(s), may be combined in advance, with the active(s) added just prior to formation of the film. This is especially important for actives that may degrade or lose their intended activity with prolonged exposure to water, air or another polar solvent. For example, some drugs, bioeffecting agents and diagnostic reagents among other actives, may hydrolyze or oxidize if left too long in the flowable film-forming matrix.

Mixing techniques may play a role in manufacturing of a pharmaceutical film which is suitable for commercialization and regulatory approval. For example, if gas, such as air, is trapped in the composition during the mixing process (or later during the film making process), it can leave voids in the film product as the liquid carrier evaporates during the drying stage. This may result in film collapse around the voids, which causes an uneven film surface and ultimately, attributes to a non-uniform final film product which may have inconsistent properties and component distribution. Uniformity of the resultant film may even be compromised even if the voids in the film caused by gas bubbles do not collapse, that is the presence of the gas bubbles themselves may cause a lack of uniformity in the film. This situation also provides a non-uniform film in that the spaces, which are not uniformly distributed, are occupying area that would otherwise be occupied by the film composition. Once uniformity in the uncut film is compromised, having a consistent dosage of active from one dosage unit to another is much more difficult to achieve. For this reason, the present invention seeks to provide a more uniform film composition through the reduction of, or complete elimination of, gas bubbles during the compounding (mixing) and the film-forming process. In a desired embodiment, the present invention provides a film forming matrix that is at least 95% free of gas bubbles, and more desirably at least 99% free of gas bubbles. In an ideal situation, the film forming matrix will be 100% free of gas bubbles, but it is understood that a minimal amount of gas bubbles may be present in the film forming matrix. A film forming matrix that is about 98 to about 99% free of gas bubbles will be referred to as “substantially free” of gas bubbles. Similarly, a resulting film product that is about 98 to about 99% free of gas bubbles will be referred to as “substantially free” of gas bubbles.

In addition, the comparison of density of the film-forming matrix before and after the deaeration step may be useful in determining whether the film-forming matrix is sufficiently deaerated. After the film-forming matrix has undergone the deaeration step (i.e., the matrix has been fed through a “volume reduction device” as explained herein), the resulting matrix should have an increased density.

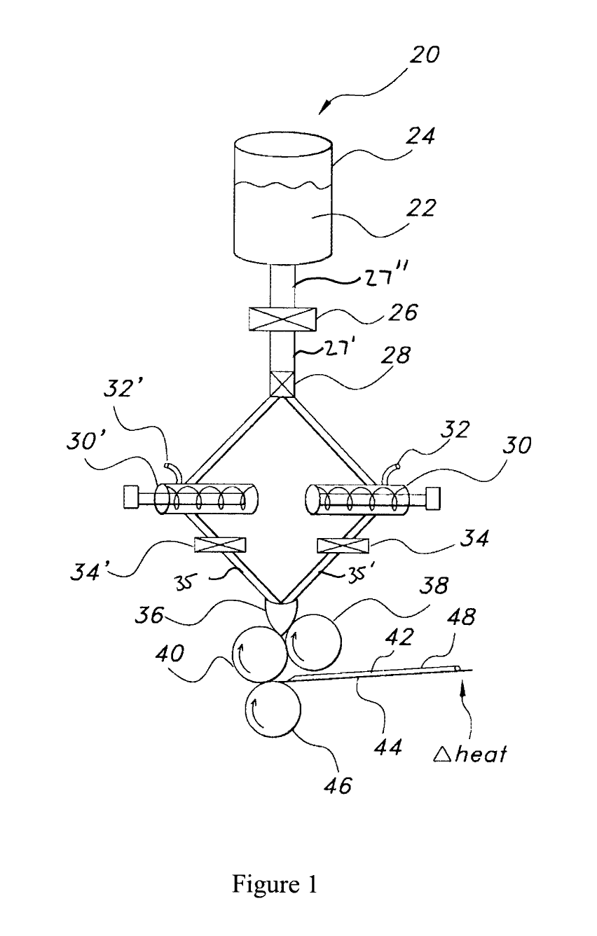

FIG. 1 shows an apparatus 20 suitable for the preparation of a pre-mix, addition of an active to the pre-mix, and the subsequent formation of a film product. The pre-mix or “master batch” 22 includes various components of the film forming matrix, including those components that are not easily degradable. Such components present in the pre-mix 22 may include, fore example, the film-forming polymer, a solvent or flowable carrier, and any other additives (such as fillers, sweeteners, and the like). The pre-mix 22 thus includes most of the components except for the active (or actives). The pre-mix 22 may be added to a master batch feed tank 24.

The components for pre-mix or master batch 22 are desirably formed in a mixer (not shown) prior to their addition into the master batch feed tank 24. Then a pre-determined amount of the master batch may be controllably fed via a first metering pump 26 and through control valve 28 to either or both of the first and second mixers, 30, 30′. The present invention, however, is not limited to the use of two mixers, 30, 30′, and any number of mixers may suitably be used. Moreover, the present invention is not limited to any particular sequencing of the mixers 30, 30′, such as parallel sequencing, and other sequencing or arrangements of mixers, such as series or combination of parallel and series, may suitably be used.

In one embodiment of the present invention, the apparatus 20 may include one or more degassing units 27 between the master batch feed tank 24 and the mixers 30, 30′. For example, a degassing unit 27′ may be disposed between the metering pump 26 and the control valve 28. In another embodiment, a degassing unit 27″ may be disposed between the master batch feed tank 24 and the metering pump 26. Any degassing type of apparatus may be used, including those described below.

The components of the film forming matrix that may be susceptible to degradation may be added to the mixer(s) separately from the pre-mix 22. The desired amount of the active or other degradable component, such as a flavor, may be added to the desired mixer through an opening, 32, 32′, in each of the mixers, 30, 30′. At this point, each of the components of the film are present in the mixers 30, 30′, where they are mixed together to form a desired film forming mixture. Desirably, the residence time of the pre-mix 22 is minimized in the mixers 30, 30′. While complete dispersion of the active into the pre-mix 22 is desirable, excessive residence times may result in leaching or dissolving of the drug, especially in the case for a soluble active. Thus, the mixers 30, 30′ are often smaller, with lower residence times required to achieve the desired level of mixing, as compared to the primary mixers (not shown) used in forming the pre-mix 22.

After the degradable component(s), including an active, has been blended with the master batch pre-mix 24 for a sufficient time to provide uniformity of drug content throughout the matrix, the matrix may then fed to a pan 36 through the second metering pumps, 34, 34′. If desired, the apparatus 20 may include a degassing unit 35 between the mixer(s) 30 and the pan 36. For example, a degassing unit 35, 35′ may be disposed between the metering pumps 34, 34′ and the pan. Alternatively, a degassing unit 35, 35′ may be disposed between the mixers 30, 30′ and the metering pumps 34, 34′. In another embodiment, there may be a first degassing unit 35, 35′ disposed between the mixers 30, 30′ and metering pumps 34, 34′, and a second degassing unit 35, 35′ disposed between the metering pumps 34, 34′ and pan. Desirably, each line from a mixer 30 through metering pump 34 and to the pan 36 includes a degassing unit 35. That is, in embodiments incorporating a degassing unit 35 after the mixer 30, each mixed film forming matrix will be degassed prior to being fed into the pan. A metering roller 38 determines the thickness of wet film forming matrix 42 and applies it to an application roller 40. The metering roller 38 may be adjusted to form a very thin film, a thick film, or any other variations as may be desired. Once the wet film 42 is formed on substrate 44, it may be carried away or conveyed onto further processing via a support roller or other means to carry the formed film.

By the time the wet film 42 is deposited onto the substrate 44, it has desirably been fed through at least one degassing unit. In some embodiments, there may be one degassing unit, such as a degassing unit 27″ disposed between the master batch feed tank 24 and the metering pump 26, or degassing unit 27′ between the metering pump 26 and control valve 28, or degassing unit 25 between the mixer 30 and pan 36. There may be more than one degassing unit in the assembly 20, at any or all of the aforementioned locations. Other degassing units may be disposed at various locations in the assembly as desired.

The combination of the multi-component matrix, which includes the polymer, water, and an active (as well as other components as desired), may be formed into a sheet or film using other equipment, instruments, or techniques besides those depicted in FIG. 1.

In addition to the aforementioned, any method known in the art such as extrusion, coating, spreading, casting or drawing the multi-component matrix may be used to form the film or sheet. Although a variety of different film-forming techniques may be used, it is desirable to select a method that will provide a flexible film, such as reverse roll coating. The flexibility of the film allows for the web of film to be rolled and transported once formed. Thus, the rolls may be stored for a period of time prior to being cut, or may be easily transported across a room or facility. Desirably, the films will also be self-supporting or able to maintain their integrity and structure in the absence of a separate support. Furthermore, the films of the present invention may be selected from materials that are edible, ingestible, biodegradable, biocompatible, and or pharmaceutically acceptable.

Multi-layered films or sheets may be formed by co-extruding more than one combination of components (of the same or different combination), or by a multi-step coating, spreading, casting, drawing, or combinations thereof. As another example, a multi-layered film may also be achieved by coating, spreading, or casting a combination onto an already formed film layer.

Coating or casting methods are particularly useful for the purpose of forming the films of the present invention. Specific examples of forming the film may include: (1) reverse roll coating; (2) gravure coating; (3) immersion or dip coating; (4) metering rod or meyer bar coating; (5) slot die or extrusion coating; (6) gap or knife over roll coating; (7) air knife coating; (8) curtain coating; or combinations thereof. Combinations of one or more of the aforementioned may be employed when the formation of a multi-layered film is desired.

Roll coating, or more specifically reverse roll coating, is particularly desired when forming films in accordance with the present invention. This procedure provides excellent control and uniformity of the resulting films, which is desired in the present invention. In this procedure, the coating material is measured onto the applicator roller by the precision setting of the gap between the upper metering roller and the application roller below it. The coating is transferred from the application roller to the substrate as it passes around the support roller adjacent to the application roller. Both three roll and four roll processes are common.

The gravure coating process relies on an engraved roller running in a coating bath, which fills the engraved dots or lines of the roller with the coating material. The excess coating on the roller is wiped off by a doctor blade and the coating is then deposited onto the substrate as it passes between the engraved roller and a pressure roller. Offset Gravure is common, where the coating is deposited on an intermediate roller before transfer to the substrate.

In the simple process of immersion or dip coating, the substrate is dipped into a bath of the coating, which is normally of a low viscosity, to enable the coating to run back into the bath as the substrate emerges.

In the metering rod coating process, an excess of the coating is deposited onto the substrate as it passes over the bath roller. The wire-wound metering rod, sometimes known as a Meyer Bar, allows the desired quantity of the coating to remain on the substrate. The quantity is determined by the diameter of the wire used on the rod.

The gap or knife over roll process relies on a coating being applied to the substrate which then passes through a “gap” between a “knife” and a support roller. As the coating and substrate pass through, the excess is scraped off.

Slot die coating is a process by which the coating solution is metered through a uniform slit by a volumetric metering pump onto the substrate. Because the metering pump is a volumetric pump, the solution density (and as such, the air bubble content) will determine the amount of coating solution on the substrate for a given pump setting. Variability in density throughout the run will then result in variability in the content of active in the final product.

Air knife coating is where the coating is applied to the substrate and the excess is “blown off” by a powerful jet from the air knife. This procedure is useful for aqueous coatings.

In the curtain coating process, a bath with a slot in the base allows a continuous curtain of the coating to fall into the gap between two conveyors. The object to be coated is passed along the conveyor at a controlled speed and so receives the coating on its upper face.

While viscosity, uniformity, stability, and casting method are important aspects of the film formation process, the method of removing the moisture from the wet film to create a dried product is also an important factor. That is, a quick, controlled drying process ensures that the uniformity which is rapidly achieved will be maintained until the film is dry.

Once the film strip is formed, the remaining water or aqueous components of the wet film are desirably removed in order to provide a final product which is in a self-supporting condition which may maintain a certain shape or conformation. Further, the active component or agent may desirably be evenly or uniformly distributed throughout the film strip product. In order to promote an exact dosing of active component or agent in each film strip, it may be desirable to make each film strip uniform in surface and consistency. As such, it may be desirable to control one or more processing parameters in order to ensure that gas bubbles, ridges, and or pockets may be reduced or altogether eliminated prior to and during the film formation and the drying process (if any) employed therewith.

### Processing the Wet Film Product to Remove Excess Moisture

The films of the present invention may contain components that are sensitive to temperature, such as flavors, which may be volatile, or drugs, which may have a low degradation temperature. In such cases, the drying techniques may be varied in order to adequately dry the uniform films of the present invention. Drying the wet film product may be desirable in order to remove excess moisture from the film product. A drying step may reduce the amount of time that a wet film is potentially exposed to contaminants, and the amount of time from processing to packaging (i.e. a more efficient manufacturing process). Excess water, solvent, or moisture in the film product may contribute to a non-uniform product and/or degradation of active components within the film or sheet. Drying may be through the evaporation of excess water at ambient or other desired temperatures over a length of time. The film may be dried at low or negative pressures (i.e. vacuum dried), or the film may be dried by air blowers, fans, and the like. The drying step may reduce any aggregation or conglomeration of the film components as it is formed into a solid structure. The drying process may further permit exposure of the film to temperatures above that at which the active component typically would degrade without loss of a desired level of activity. It is understood, of course, that the temperature outside the film forming matrix may be higher than the temperature within the film forming matrix, such that the matrix is heated to a temperature at which the active is not degraded.

The wet film may then be dried using controlled bottom drying or controlled microwave drying, desirably in the absence of external air currents or heat on the top (exposed) surface of the film 48 as described herein. Controlled bottom drying or controlled microwave drying advantageously allows for vapor release from the film.

Conventional convection air drying from the top is not preferably employed as it initiates drying at the top uppermost portion of the film, thereby forming a barrier against fluid flow, such as the evaporative vapors, and thermal flow, such as the thermal energy for drying. Such dried upper portions serve as a barrier to further vapor release as the portions beneath are dried, which results in non-uniform films. As previously mentioned, some top air flow can be used to aid the drying of the films of the present invention, but it preferably does not create a condition that would cause particle movement or a rippling effect in the film, both of which would result in non-uniformity. If top air is employed, it is preferably balanced with the bottom air drying to avoid non-uniformity and prevent film lift-up on the carrier belt. A balanced top and bottom air flow may be suitable where the bottom air flow functions as the major source of drying and the top air flow is the minor source of drying. The advantage of some top air flow is to move the exiting vapors away from the film thereby aiding in the overall drying process. The use of any top air flow or top drying, however, is preferably be balanced by a number of factors including, but not limited, to rheological properties of the composition and mechanical aspects of the processing. Any top fluid flow, such as air, also preferably does not overcome the inherent viscosity of the film-forming composition. In other words, the top air flow cannot break, distort or otherwise physically disturb the surface of the composition. Moreover, air velocities are desirably below the yield values of the film, i.e., below any force level that can move the liquids in the film-forming compositions. For thin or low viscosity compositions, low air velocity must be used. For thick or high viscosity compositions, higher air velocities may be used. Furthermore, air velocities are desirably low so as to avoid any lifting or other movement of the film formed from the compositions.

In bottom drying, the evaporating vapors more readily carry heat away from the film as compared to top drying which lowers the internal film temperature. Such lower internal film temperatures often result in decreased drug degradation and decreased loss of certain volatiles, such as flavors.

During film preparation, it may be desirable to dry films at high temperatures. High heat drying produces uniform films, and leads to greater efficiencies in film production. Films containing sensitive active components, however, may face degradation problems at high temperatures. Degradation is the “decomposition of a compound . . . exhibiting well-defined intermediate products.” The American Heritage Dictionary of the English Language (4^{th} ed. 2000). Degradation of an active component is typically undesirable as it may cause instability, inactivity, and/or decreased potency of the active component. For instance, if the active component is a drug or bioactive material, this may adversely affect the safety or efficacy of the final pharmaceutical product. Additionally, highly volatile materials will tend to be quickly released from this film upon exposure to conventional drying methods.

Degradation of an active component may occur through a variety of processes, such as, hydrolysis, oxidation, and light degradation, depending upon the particular active component. Moreover, temperature has a significant effect on the rate of such reactions. The rate of degradation typically doubles for every 10° C. increase in temperature. Therefore, it is commonly understood that exposing an active component to high temperatures will initiate and/or accelerate undesirable degradation reactions.

During the drying process of the present invention, several factors produce uniformity within the film while maintaining the active component at a safe temperature, i.e., below its degradation temperature. First, the films of the present invention have an extremely short heat history, usually only on the order of minutes, so that total temperature exposure is minimized to the extent possible. Second, the films are controllably dried to prevent aggregation and migration of components, as well as preventing heat build up within. Third, the films are desirably dried from the bottom, as controlled bottom drying, as described herein, prevents the formation of a polymer film, or skin, on the top surface of the film. As heat is conducted from the film bottom upward, liquid carrier, e.g., water, rises to the film surface. The absence of a surface skin permits rapid evaporation of the liquid carrier as the temperature increases, and thus, concurrent evaporative cooling of the film. Due to the short heat exposure and evaporative cooling, the film components such as drag or volatile actives remain unaffected by high temperatures. In contrast, skinning on the top surface traps liquid carrier molecules of increased energy within the film, thereby causing the temperature within the film to rise and exposing active components to high, potentially deleterious temperatures.

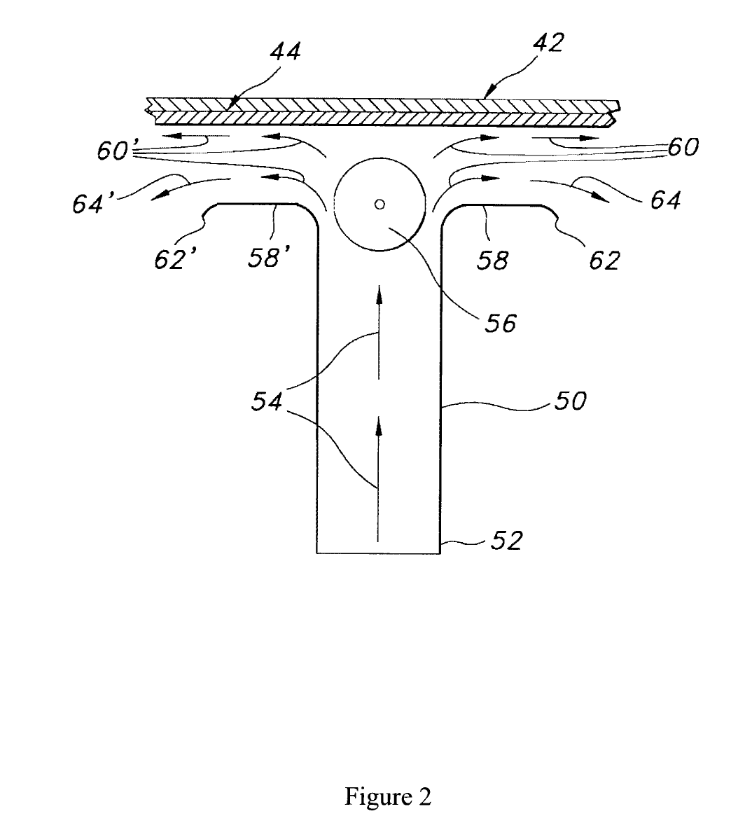

Although the inventive process is not limited to any particular apparatus for the above-described desirable drying, one particular useful drying apparatus 50 is depicted in FIG. 2. Drying apparatus 50 is a nozzle arrangement for directing hot fluid, such as but not limited to hot air, towards the bottom of the film 42 which is disposed on substrate 44. Hot air enters the entrance end 52 of the drying apparatus and travels vertically upward, as depicted by vectors 54, towards air deflector 56. The air deflector 56 redirects the air movement to minimize upward force on the film 42. As depicted in FIG. 2, the air is tangentially directed, as indicated by vectors 60 and 60′, as the air passes by air deflector 56 and enters and travels through chamber portions 58 and 58′ of the drying apparatus 50. With the hot air flow being substantially tangential to the film 42, lifting of the film as it is being dried is thereby minimized. While the air deflector 56 is depicted as a roller, other devices and geometries for deflecting air or hot fluid may suitable be used. Furthermore, the exit ends 62 and 62′ of the drying apparatus 50 are flared downwardly. Such downward flaring provides a downward force or downward velocity vector, as indicated by vectors 64 and 64′, which tend to provide a pulling or drag effect of the film 42 to prevent lifting of the film 42. Lifting of the film 42 may not only result in non-uniformity in the film or otherwise, but may also result in non-controlled processing of the film 42 as the film 42 and/or substrate 44 lift away from the processing equipment.

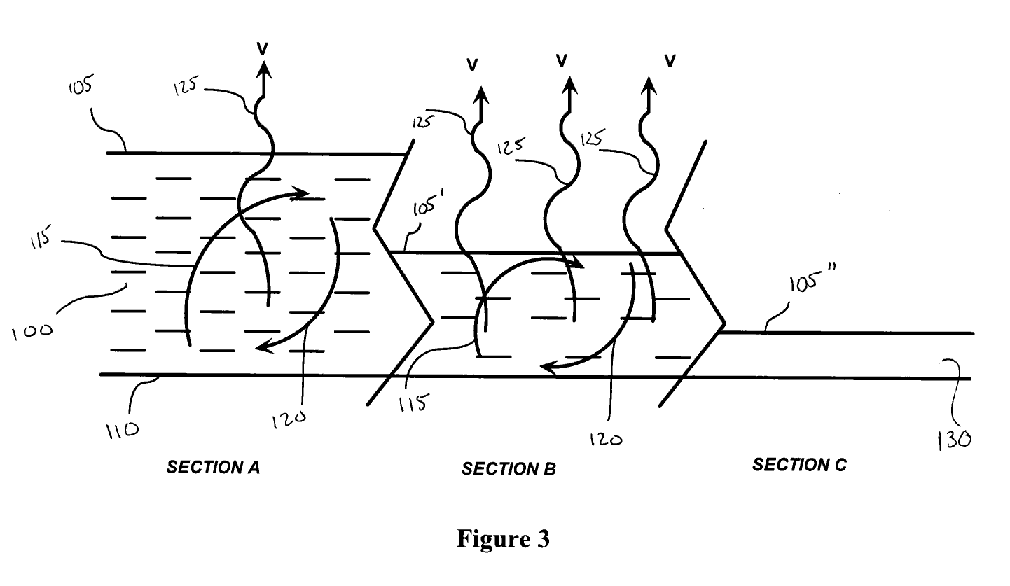

FIG. 3 is a sequential representation of one drying process useful in the present invention. After mechanical mixing, a wet film matrix 100 may be placed on a conveyor for continued thermal mixing during the drying process. At the outset of the drying process, depicted in Section A, the wet film forming matrix 100, having top surface 105 and bottom surface 110, is heated, optionally from the bottom 110, as it is travels via conveyor (not shown). Heat may be supplied to the film by any desired heating mechanism.

As the film is heated, any liquid carriers, or volatiles (“V”) present in the wet film, begin to evaporate, as shown by upward arrow 125. Thermal mixing is a fairly cyclic process, in which hotter liquid, depicted by arrow 115, rises and cooler liquid, depicted by arrow 120, takes its place. This allows the film to dry in a controlled manner, while volatiles are evaporated from the top surface 105 of the wet film matrix 100. Further, since there is no skin formation on the top surface 105 of the wet film 100, (as shown in Section B), any volatile liquid continues to evaporate 125 out the top surface 105′ of the wet film matrix 100. Additionally, during this stage in the process, thermal mixing 115/120 continues to distribute controlled and even thermal energy throughout the wet film 100. Once a sufficient amount of the volatile liquid has evaporated 125, thermal mixing (115/120) will have produced uniform heat diffusion throughout the wet film forming matrix 100. The resulting dried film 130 is a visco-elastic solid having a top surface 105″, as depicted in Section C. At this point in the drying process, the components of the visco-elastic solid 130 are locked into place in a uniform distribution throughout the film 130. Minor amounts of liquid carrier, i.e., water or solvent, may remain subsequent to formation of the visco-elastic solid 130. Although minor amounts of liquid carrier, i.e., water or solvent, may remain subsequent to formation of the visco-elastic 130, the film may be dried further without movement of the particles, if desired. As can be seen, during the drying process (i.e., from Section A to Section C), the thickness of the matrix is reduced, due to evaporation of the volatiles present in the matrix 100.

The drying step(s) remove the liquid carriers from the film in a manner such that the uniformity, or more specifically, the non-self-aggregating uniform heterogeneity, that is obtained in the wet film 100 is maintained until the visco-elastic mass 130 is formed. The temperature of the oven, the length of drying time and the amount of humidity in the ambient air may be controllable factors in the drying process. The amount of energy, temperature and length and speed of the conveyor can be balanced to accommodate such actives and to minimize loss, degradation or ineffectiveness in the final film. Desirably, the drying oven (or ovens) is first turned on and is allowed to run until the temperature within the oven has stabilized at the set point before coating is started. The length of the drying time may be altered as necessary to achieve the drying desired. For example, when a smaller batch size is used, or when the coating is narrow, the speed of the product through the drying oven may be increased, thus reducing the drying time. The drying time may be changed via the speed at which the film travels, or the number of ovens through which the film travels. For example, in one embodiment, the drying process includes passing the film through at least two oven segments (or “zones”), or at least five oven segments (“zones”). Any number of oven segments may be used in the drying process to achieve the desired film.

Monitoring and control of the thickness of the film also contributes to the production of a uniform film by providing a film of uniform thickness. The thickness of the wet film 100 may be monitored with gauges, such as Beta or Gamma Gauges. A gauge may be coupled to another gauge at the end of the drying apparatus, i.e. drying oven or tunnel, to communicate through feedback loops to control and adjust the opening in the coating apparatus, resulting in control of uniform film thickness. Desirably, the film is formulated so that the dimensional changes incurred during drying are to the film's thickness and not its width. As such, monitoring of the film's thickness may be helpful in maintaining a suitable product.

### Cutting and Packaging the Film Product

Once the product is mixed, formed, and dried into a thin film or roll product, the film or roll of film may be cut into certain shapes, dimensions, etc. and packaged in a desirable contaminant-preventing and shelf-life promoting packaging material. In the cutting process, the equipment may generally include a slitter and a mounted package machine

### Deaeration/Degassing of The Film-Forming Matrix

During production and manufacturing of the film, the various components are mixed in one or more mixers, as previously discussed. As discussed above, the assembly 20 desirably includes one or more degassing units (including degassing units 27 and 35). These degassing units desirably create a film forming matrix (or pre-mix, if appropriate) that is substantially free of gas bubbles. Again, the degassing unit may be used to reduce/eliminate gas bubbles in a pre-mix, after the active has been added, or both. Degassing of the film forming matrix may be evaluated through any desired means so as to determine whether the film forming matrix has been sufficiently degassed. In one embodiment, the effectiveness of the degassing step may be determined through a measurement of the percentage of gas bubbles in the matrix. As explained above, in a desired embodiment, the present invention provides a film forming matrix that is at least 95% free of gas bubbles, and more desirably at least 99% free of gas bubbles. In an ideal situation, the film forming matrix will be 100% free of gas bubbles, but it is understood that a minimal amount of gas bubbles may be present in the film forming matrix. A film forming matrix that is about 98 to about 99% free of gas bubbles will be referred to as “substantially free” of gas bubbles. Similarly, a resulting film product that is about 98 to about 99% free of gas bubbles will be referred to as “substantially free” of gas bubbles.

In another embodiment, the effectiveness of the degassing step may be determined by a comparison of the density of the film-forming matrix before and after the degassing step. That is, prior to the step of degassing the matrix (regardless of whether the matrix includes actives or does not include actives), the non-degassed matrix has a first density.

Once the film-forming matrix has traveled through the volume reduction device, and thus, after degassing, the resultant degassed matrix has second density, based on the geometry of the device. As can be appreciated by one of skill in the art, this second density will be higher than the first density in order to facilitate degassing. In desired embodiments, the second density may be about 1 to about 10 times higher than the first density.

The volume reduction device of the present invention may effectively and efficiently treat a masterbatch premix, a film-forming matrix, a film-forming and active-containing matrix, and combinations thereof in order to reduce the amount of, or altogether remove gas bubbles from the various fluids. The resulting degassed matrix is desirably substantially free of gas bubbles. As a result, the film-forming composition has a high uniformity of content such that, when cast, the films are continuous and do not exhibit substantial aberrations therein from gas bubbles that may become entrapped in the film. Thus, dosages are consistently uniform in drug content from one dosage to the next, from one batch to the next.

The volume reduction device (which may be a degassing device) may take one or more various forms. Certain desirable characteristics are shared by the various forms of the volume reduction device. The volume reduction device may promote the flow of the fluid to be degassed into a thin, spread out fluid flow path.

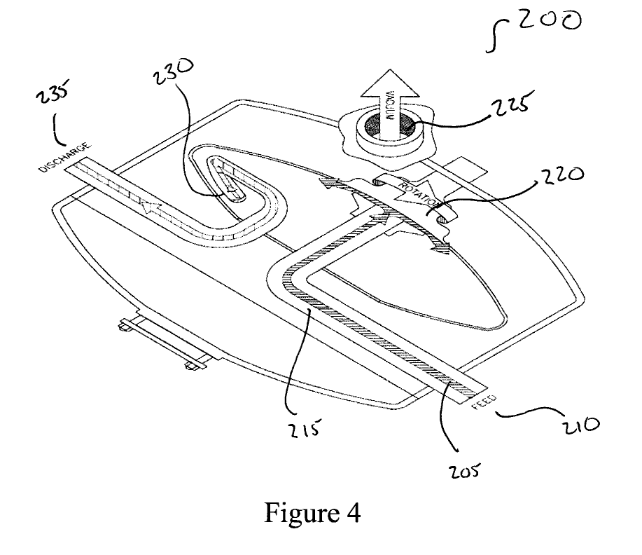

One useful volume reduction device, as shown in FIG. 4, is one sold under the trade name Versator 200 (sold by Cornell). The Versator is generally available in three sizes, a small, medium and large size. The size of the apparatus 200 may vary, and may be selected by the user to suit the level of material that is desired to be dearated. The principle of operation for the Cornell Versator is the same for all three (3) sizes of Versator. During operation, a film-forming matrix 205 is drawn into the Versator chamber via input port 210 under vacuum. The film forming matrix 205 travels through the feed assembly 215 towards the center of the rotating Versator Disc 220. At the end of the feed assembly, a spreader ring spreads the film-forming matrix 205 into a thin film on the surface of the Versator Disc 220. Centrifugal forces then drive the film-forming matrix 205 towards the Versator Disc's outer edge. While in transit, the rotation of the Versator Disc 220 combines with the vacuum in the Versator Chamber 225 to create a combination of shear, turbulence, and frictional force on the film-forming matrix 205. Within fractions of a second, the film-forming matrix 205 is constantly thinned, and any entrapped bubbles are drawn to the film's surface and broken. A scoop tube assembly 230 then picks up the film-forming matrix 205 at the outer edge of the Versator Disc 220 and discharges it from the Versator chamber through output port 235.

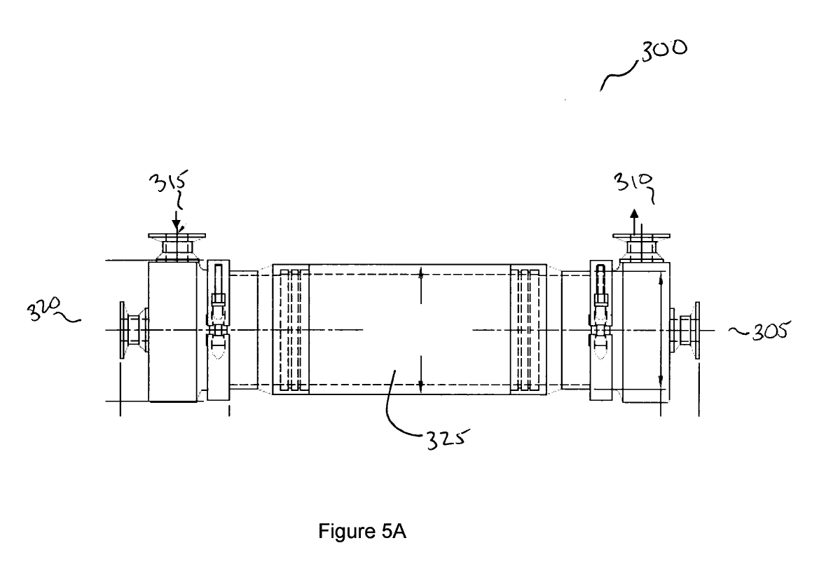

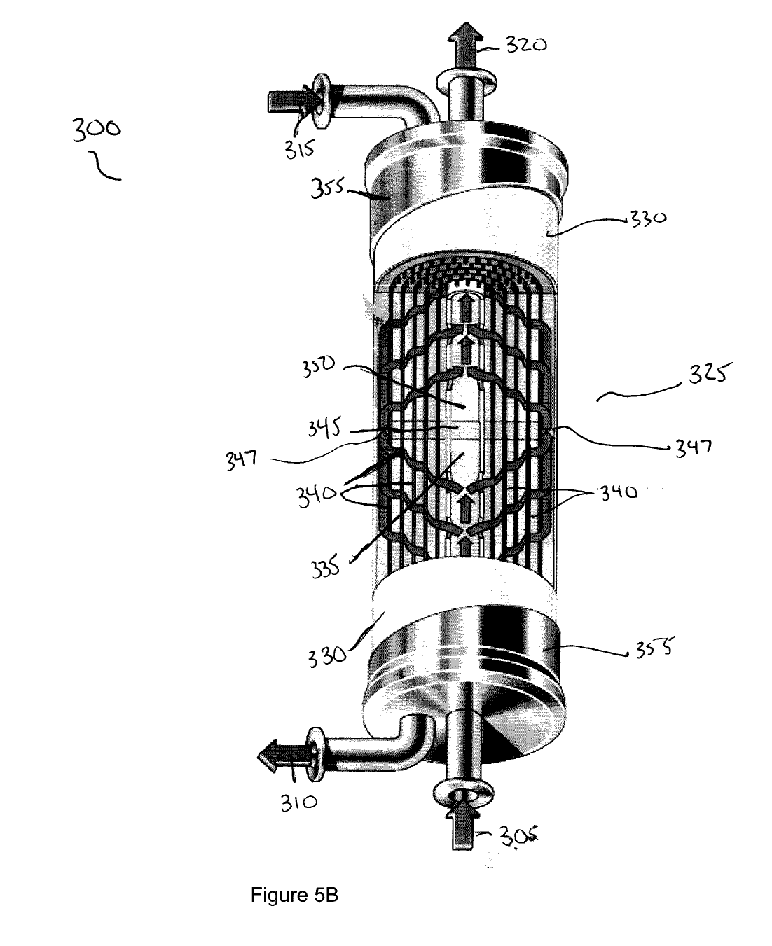

Another useful volume reduction device, as shown in FIGS. 5A and 5B, is referred to as a membrane contactor 300. In use, the film forming matrix 205 may flow through one or more membrane contactors 300. As with the Versator 200, a membrane contactor 300 may be used at any desired location in the device, including, for example, after the master batch feed tank 24, after the pump 26, after the valve 28, after the mixer 30, or after the pump 34 (before feeding into pan 36). The membrane contactor 300 may be used to deaerate the pre-mix composition, the film forming composition (with active), or both. Membrane contactors 300 may be more efficient than vacuum towers and forced draft deaerators, and additionally may be capable of working inline with the processing parameters, equipment and instruments.

As can be seen in FIG. 5A, a membrane contactor 300 generally includes an inlet port 305, for introducing the film forming matrix 205. The membrane contactor 300 also includes a means for introducing a vacuum into the contactor 300. In a desired embodiment, the contactor 300 includes a vacuum outlet port 310 and a vacuum inlet port 315. Desirably, the vacuum outlet port 310 is disposed at a location closer to the fluid inlet port 305. At a location opposite from the inlet port 305, there is a fluid output port 320. Between the fluid inlet port 305 and the fluid output port 320 is the contactor body 325, which will be described in further detail below. In use, the film forming matrix 205 is introduced into the inlet port 305, travels through the contactor body 325, and out the output port 320. During the travel through the contactor body 325, the film forming matrix 205 is deaerated, and thus a deaerated matrix flows out the output port 320.

FIG. 5B is a cross-sectional view of one embodiment of a membrane contactor 300 useful in the present invention. In this embodiment, the contactor 300 has a generally cylindrical shape, but it will be understood that the contactor 300 may have a non-rounded shape, including a box-like shape, or other shape. At the first end of the contactor 300, there is disposed an inlet port 305 for introducing a film forming matrix 205. The film forming matrix 205, when introduced into the contactor 300, will be referred to as an “aerated matrix”. It will, of course, be understood that the aerated matrix may be subjected to a deaeration treatment before introduction into the contactor 300. For example, it may be desired to use a series of deaeration devices to ensure full and complete deaeration. An aerated matrix may include a film forming matrix that has not been treated with any deaeration method, or the aerated matrix may have been treated with a deaeration method prior to introduction into the contactor 300.

At the second end of the contactor 300, which is located opposite from the inlet port 305, there is disposed an output port 320, through which the film forming matrix 205 exits the contactor 300 after undergoing deaeration. For purposes herein, after exiting the contactor 300 the film forming matrix 205 will be referred to as a “deaerated matrix”. It will be understood that the deaerated matrix may be only partially deaerated, and thus it may be desired that the deaerated matrix be introduced into another deaeration method (such as a second contactor 300 or a versator 200). It is preferred that the deaerated matrix be at least 99% deaerated, but a “deaerated matrix” refers to a film forming matrix 205 that has undergone at least one deaeration method.

The contactor 300 includes a vacuum introduction means, generally depicted as a vacuum outlet port 310 and a vacuum inlet port 315. The vacuum inlet port 315 is desirably disposed at a location near the fluid output port 320, and the vacuum outlet port 310 is desirably disposed at a location near the fluid inlet port 305, but the ports 310, 315 may be located at any region of the contactor 300 desired. As discussed above, the contactor 300 includes a contactor body 325, through which the film forming matrix 205 flows and becomes deaerated. The contactor body 325 includes a cartridge 330, which spans the length of the contactor body 325 from the fluid inlet port 305 to the fluid output port 320. Within the cartridge 330 is a central distribution tube 335, along which the film forming matrix 205 is designed to flow. Around the central distribution tube 335 are a plurality of longitudinal hollow fiber membranes 340, which desirably span the length of the contactor body 325. The hollow fiber membranes 340 are made of hollow fibers, between which the film forming matrix 205 may flow. The hollow fiber membranes 340 act as porous deaerating channels. That is, as the film forming matrix 205 flows between the hollow fiber membranes 340, the film forming matrix 205 is deaerated.

At a location near the center of the contactor body 325 is a baffle 345, which is designed to restrict the flow of the film forming matrix 205. The baffle 345 spans a majority of the center of the contactor body 325, but does not reach the outer edge of the contactor body 325. In this fashion, any film forming matrix 205 that flows from the inlet port 305 to the output port 320 is directed from the center of the cartridge 330 towards the outer radius of the cartridge 330, and thus between a plurality of hollow fiber membranes 340. The film forming matrix 205 passes the baffle 345 at the baffle outside region 347.

Once the film forming matrix 205 has passed through the baffle outside region 347, the film forming matrix 205 is directed towards the center of the cartridge 330 to collection tube 350. Collection tube 350 is a substantially longitudinal tube which spans from the baffle 345 to the output port 320. After the film forming matrix 205 passes the baffle 345, it is directed towards the collection tube 350, and thus passes between a plurality of hollow fiber membranes 340. The collection tube 350 directs the film forming matrix 205 to the output port 320, where the deaerated matrix may exit the contactor 300. The contactor 300 may include an impermeable external housing 355 surrounding the cartridge 330. As the film forming matrix 205 passes between the plurality of hollow fiber membranes 340, the matrix 205 is deaerated.

The Versator 200 and the membrane contactor 300 may both be referred to as “deaeration devices”, since these devices allow for deaerating of the film forming matrix in a reduced deaeration time with a more effective deaeration as compared to conventional methods. Further, it has been found that a film forming matrix 205 that is deaerated in the above methods has a sustained quality of blend and mixture of components prior to and after the deaeration step(s) are achieved. In addition, the present methods of deaeration allow for an effective dearating of the film forming matrix without unintentional or inadvertent removal of solvent, which may occur in other methods. As such, the film forming matrix 205 is deaerated more efficiently and effectively through the present invention as compared to other conventional methods.

It will be understood that, in addition to deaerating the film forming matrix, the deaeration step may also include defoaming, degassing, debubbling, and/or homogenization of the blend of components. Deaeration may be performed on the pre-mix, on the film forming composition with active, or on both. Further, the film forming matrix 205 may be deaerated through a series of deaeration methods, such as by passing through a versator 200 and a membrane contactor 300, or through a series of versators 200, or through a series of membrane contactors 300, or any combination thereof. The resulting deaerated matrix is desirably at least 95% free of entrapped gas, and more desirably is at least 99% free of entrapped gas. In the most desirable embodiment, the resulting deaerated film forming matrix is 100% free of entrapped gas. The deaerated film forming matrix is preferably “substantially” free of entrapped gas. As used herein, the term “substantially free” of entrapped gas refers to a film forming matrix that is at least 99% free of entrapped gas.

Advantages of deaeration include, for example, an effective removal of entrapped air, foam, or gas to promote a uniform dispersion of active in the film-forming matrix (i.e., uniformity of active content per volume of film), and a uniform final product. The present invention provides an efficient, in-line step, which may be completed in reduced time than other traditional degassing/deaerating steps, including conventional vacuum and/or suction. The present invention further provides an easy to use apparatus and method for deaerating the film forming matrix at various locations and times during the various mixing stages and/or prior to coating/casting, giving the user the freedom to choose when and where to deaerate. The deaerating step may be completed at various stages in the process, as shown in FIG. 1. For example, the in-line deaeration step may take place in the pre-mix mixers (not shown), in-line between the master batch feed tank mixer 24 and the secondary mixers 30, 30′ (daughter mixers), either before or after the pump 26 (such as at locations 27′ and 27″), and/or after the secondary mixers 30, 30′ prior to the coating pan 36 (such as at location 35). The in-line deaeration step may occur at one or more of these locations. Further the deaeration step may occur in the mixer or in the hoses as the film-forming matrix is headed to the coater, such that the deaeration step may be in-line with the rest of the manufacturing process. In addition, the apparatus 20 may include a feedback line, which leads the film forming matrix 205 back into the mixing apparatus 20 after deaeration. In some embodiments, the feedback line may include an in-line deaeration method (i.e., a versator or membrane contactor) housed therein.

Deaerating the film-forming matrix results in a predicable composition of the final product, proper quality, and better uniformity of components.

The present invention is capable of working on materials at any desired temperature, whether heated, cooled or at ambient temperature. Further, the present invention is capable of achieving deaeration on a continuous basis, a semi-continuous basis (e.g., via deaerating in a plurality of small batches run continuously), or in batch processing, as desired. The present invention is beneficial in that it promotes minimal loss of product, and has minimal parts that are subject to wear and tear or likelihood of defect.

While various processing parameters have been discussed in the aforementioned paragraphs, the present inventors have determined various advantageous characteristics for performing the deaeration step, which results in a final product, which has been controlled to limit and or reduce the amount of gas that is in the final liquid and/or aqueous product. While particular embodiments of the present invention have been described herein for purposes of illustration, many modifications and changes will become apparent to those skilled in the art. Accordingly, the appended claims are intended to encompass all such modifications and changes as fall within the true spirit and scope of this invention.

### EXAMPLES

### Example 1

### Formation of Throat Film, Product Deaerated Using Membrane Contactor

In this Example, the particular equipment used included a 250 gallon mix tank, which fed into a lobe pump, which is fed through a membrane contactor and into the final vessel. An 850 kg batch of film-forming matrix (in solution form) was mixed without degassing. The matrix was pumped through the membrane contactor. The rate at which the matrix was pumped was relatively low, kept at less than 0.5 kg/hr. The viscosity of the matrix used in this example was high, over 24,000 cps. The solution was observed to have a jelly-like texture that resisted free flow. Upon exiting the deaeration device, (in this example, a membrane contactor), the matrix was observed to be noticeably free from gas bubbles. Thus, the membrane contactor was determined to effectively deaerate the matrix in-line.

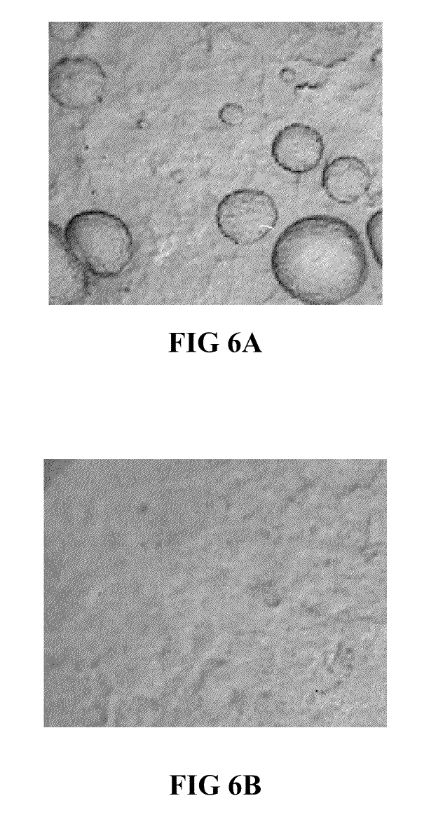

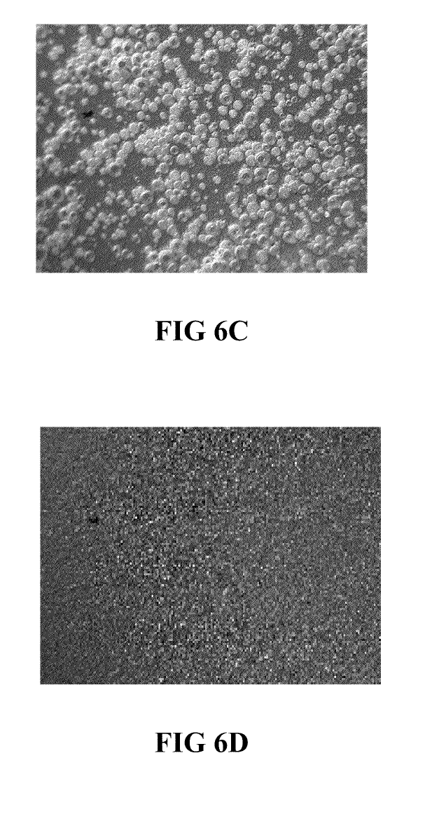

In order to more thoroughly analyze the performance of the deaeration apparatus, it was determined that another trial with a lower viscosity solution should be used to evaluate performance. Pictures were taken via microscope, both before and after deaeration of the matrix. The appearance of the solution both before and after deaeration is depicted in the Photos labeled FIG. 6A and FIG. 6B. FIG. 6A depicts the solution prior to deaeration, while FIG. 6B depicts the solution after deaeration with the Membrane Contactor. As can be seen, prior to deaeration with a membrane contactor, shown in FIG. 6A, the solution has a substantial number of gas bubbles. However, after deaeration, as can be seen in FIG. 6B, the resulting solution is substantially free of gas bubbles.

### Example 2

### Use of a D-8 Cornell Versator to Deaerate a Formulation of Throat Film

In this Example, the particular equipment used included a funnel, a Versator, a 3 horsepower lobe pump, and an open vessel. A matrix as in Example 1 was provided. Gas bubbles were purposely added to the matrix of Example 1 by mixing the solution at high speed while pulling air into the mixing head via vacuum. The resulting matrix contained a large amount of gas bubbles.

Approximately 1 gallon of aerated matrix was manually poured into a funnel attached to the Versator inlet. The lobe pump was positioned at the outlet of the Versator to relieve any back pressure. The outlet of the lobe pump opened into an open vessel.

The Versator was run at full speed, that is at about 6,000 rpm. The vacuum attached to the Versator reached a level of −28″ Hg. The Flow Rate was estimated at 7 kg/min. The matrix exiting the lobe pump was observed to be noticeably free of gas bubbles, thus evidencing that the system was effective in deaerating the matrix. Pictures were taken via microscope of the before and after appearance of the solution. Photos are depicted in FIG. 6C and FIG. 6D. FIG. 6C depicts the solution with a large number of visible gas bubbles (prior to deaeration), while FIG. 6D depicts the solution after deaeration with the Versator. As can be seen in FIG. 6D, after deaeration, the resulting film is substantially free of entrapped gas bubbles.

### Example 3

### Use of a D-8 Cornell Versator to Deaerate a Formulation of Calcium Sennosides

In this Example, the particular equipment used was similar to Example 2 above, and included a funnel, a versator, a 3 horsepower lobe pump, and an open vessel. A previously gassed sennosides solution including a suspension of a 7% suspension of Calcium Sennosides particles suspended in an aqueous polymer solution was provided. The suspension was very dark brown in color and opaque. Air bubbles were introduced into the Sennosides Solution in a similar fashion as in Example 2 above. While air was introduced into the solution, a visually noticeable amount of air (visually observable amount of bubbles) could not be observed due to the opacity of the solution.

Approximately 1 gallon of aerated sennosides solution was manually poured into a funnel attached to the Versator inlet. The lobe pump was positioned at the outlet of the Versator to relieve any back pressure. The outlet of the lobe pump opened into an open vessel.

The Versator was run at full speed, that is at about 6,000 rpm. The vacuum attached to the Versator reached a level of −28″ Hg. The Flow Rate was estimated at 7 kg/min. The user was unable to observe any visible impact on the solution exiting the lobe pump, likely due to the opacity of the suspension. This served to demonstrate that the present invention may be used as an additional degassing step in the process without any visible deleterious effects to the Calcium Sennosides suspension.

### Example 4

### Use of a D-8 Cornell Versator and Colloid Mill

In this Example, the equipment used included a 500 gallon hold tank, which was fed into a colloid mill, and then into a lobe pump, and finally into an open tank. The product used included a Peppermint flavor film forming matrix. This Example determined the feasibility of running the Versator and Colloid Mill in the same equipment train. Further, the Example served to demonstrate that the present invention may be used as an additional degassing step in the process without any visible deleterious effects to the polymer solution.

A Peppermint flavor film forming matrix was provided and degassed. The matrix was pumped through the Versator by the Colloid Mill. The Versator rate was sufficient to match any flow rate delivered by the Colloid Mill. The Colloid Mill was run at full speed (that is, about 3,600 rpm), and the flow rate was controlled by setting the gap clearance of the mill.

There was no visible difference in the matrix exiting the equipment train. Flow rates reached in excess of 10 kg/min.

### Example 5

### Use of a Colloid Mill and D-8 Cornell Versator

In this Example, the equipment used included a 250 Gallon Mix Tank, which flowed into a Colloid Mill, then into a Versator, a Lobe Pump, and into an Open Tank. A 950 kg batch of Niacinimide Stock solution including a 10% PEO polymer solution combined with assorted flavors and coloring was mixed. The Stock Solution was passed through the colloid mill and then through the Versator. The colloid mill was run at a setting of 45 Hz and a gap of 0, while the Versator was run at a speed setting of 9. The flow rate was very low due to the low gap setting of the Colloid Mill. Solution rate was approximately 1-2 kg/min. The solution exiting the Versator was visibly free from gas bubbles, evidencing its effectiveness as a deaeration system.

What is claimed is:

1. A process for preparing a degassed film-forming matrix comprising the steps of:
(a) providing a film-forming pre-mix comprising an edible polymer component and a fluid carrier selected from the group consisting of water, organic solvents, and combinations thereof;
(b) mixing the pre-mix with an active component to form a film-forming matrix; and
(c) degassing said film-forming matrix to provide a degassed matrix having an increased density, said degassing step comprising directing a flow of said film forming matrix having a first density through at least one volume reduction device,
wherein the flow through said volume reduction device increases the density of the matrix to form a matrix having a second density, said second density being higher than said first density.

2. The process of claim 1, wherein said step of degassing said film-forming matrix is performed prior to pumping said matrix through a metering pump.

3. The process of claim 1, wherein said step of degassing said film-forming matrix is performed after pumping said matrix through a metering pump.

4. The process of claim 1, further comprising the step of degassing said pre-mix prior to the step (b) of mixing the pre-mix with an active component.

5. The process of claim 1, further comprising the step of forming a wet film comprising said resultant degassed matrix wherein said active component is uniformly distributed throughout.

6. The process of claim 1, further comprising the step of determining a gas bubble content subsequent to said step of degassing, and comparing said determined gas bubble content with a predetermined acceptable gas bubble content.

7. The process of claim 6, further comprising the step of determining whether additional degassing is required based upon said comparison.

8. The process of claim 1, wherein the step of degassing is conducted at a low pressure.

9. The process of claim 1, wherein the step of degassing is conducted in a vacuum.

10. The process of claim 1, wherein the step of degassing further comprises directing the matrix through a plurality of in-series volume reduction devices.

11. The process of claim 10, wherein each of said in-series reduction devices provides a higher density than said first density of said matrix.

12. The process of claim 1, wherein said step of degassing further comprises directing the matrix through a plurality in-parallel volume reduction devices.

13. The process of claim 12, wherein each of said in-parallel reduction devices provides a higher density than said first density of said matrix.

14. The process of claim 1, further comprising the step of repeating said degassing step until a desired level of degassing is reached in said degassed matrix.

15. The process of claim 1, wherein the degassing step is performed in-line.

16. A process for preparing a self-supporting film-forming composition comprising:
(a) providing a film-forming matrix comprising an edible polymer component and a fluid carrier selected from the group consisting of water, organic solvents, and combinations thereof:
(b) degassing said matrix, comprising the steps of:
(i) directing a flow of said matrix having a first density through at least one volume reduction device;
(ii) increasing the density of the matrix to yield a flow of a resultant degassed matrix;
(c) mixing the resultant degassed matrix with an active component to form a degassed matrix.

17. The process of claim 16, wherein said step of degassing said matrix is performed prior to pumping said matrix through a metering pump.

18. The process of claim 16, wherein said step of degassing said matrix is performed after pumping said matrix through a metering pump.

19. The process of claim 16, further comprising the step of (d) degassing said degassed matrix having a uniform distribution of said active component therein.

20. The process of claim 16, further comprising the step of forming a wet film comprising said resultant degassed matrix wherein said active component is uniformly distributed throughout.

21. The process of claim 16, further comprising the step of determining a gas bubble content subsequent to degassing, and comparing said determined gas bubble content with a predetermined acceptable gas bubble content

22. The process of claim 21, further comprising the step of determining whether additional degassing is required based upon said comparison.

23. The process of claim 16, wherein the step of degassing is conducted at a low pressure.

24. The process of claim 16, wherein the step of degassing is conducted in a vacuum.

25. The process of claim 16, wherein the step of degassing further comprises directing the matrix through a plurality of in-series volume reduction devices.

26. The process of claim 25, wherein each of said in-series reduction devices provides a higher density than said first density of said matrix.

27. The process of claim 16, wherein the step of degassing further comprises directing the matrix through a plurality in-parallel volume reduction devices.

28. The process of claim 27, wherein each of said in-parallel reduction devices provides a higher density than said first density of said matrix.

29. The process of claim 16, further comprising the step of repeating the degassing step until a desired level of degassing is reached in said degassed matrix.

30. The process of claim 16, wherein the degassing step is performed in-line.

31. A process for preparing a self-supporting film-forming composition comprising:
(a) providing a film-forming matrix comprising an edible polymer component and a fluid carrier selected from the group consisting of water, organic solvents, and combinations thereof;
(b) degassing said film-forming matrix by directing a flow of said film-forming matrix having a first density through at least one volume reduction device, said device comprising a plurality of porous deaerating channels, so as to form a film-forming matrix having a second density, said second density being higher than said first density; and
(c) mixing said film-forming matrix having a second density with an active component to form an active-containing degassed film-forming matrix.

32. The process of claim 31, wherein said step of degassing said matrix is performed prior to pumping said matrix through a metering pump.

33. The process of claim 31, wherein said step of degassing said matrix is performed after pumping said matrix through a metering pump.

34. The process of claim 31, further comprising the step of (d) degassing said active-containing degassed film-forming matrix.

35. The process of claim 31, further comprising the step of forming a wet film from said active-containing degassed film-forming matrix.

36. The process of claim 31, further comprising the step of determining whether additional degassing of said active-containing degassed film-forming matrix is required.

37. The process of claim 31, wherein the step of degassing is conducted at a low pressure.

38. The process of claim 31, wherein the step of degassing is conducted in a vacuum.

39. The process of claim 31, wherein the step of degassing is performed in-line.

40. The process of claim 31, wherein the step of degassing further comprises directing the film-forming matrix through a plurality of volume reduction devices in series.

41. The process of claim 31, wherein the step of degassing further comprises directing the film-forming matrix through a plurality of volume reduction devices in parallel.

42. The process of claim 31, wherein said porous deaerating channels are formed of a hydrophobic membrane, said membrane being permeable to a gas and impermeable to a non-gaseous material.

43. A process for preparing a self-supporting film-forming composition comprising:
(a) forming a matrix including an edible polymer component and; a fluid carrier;
(b) degassing said matrix comprising the steps of: directing a flow of said matrix having a first density through at least one volume reduction device, said volume reduction device including a rotatable surface, so as to form a film-forming matrix having a second density, said second density being higher than said first density; and
(c) mixing the resultant film-forming matrix having a second density with an active component to form an active-containing degassed film-forming matrix.

44. The process of claim 43, wherein said step of degassing said matrix is performed prior to pumping said matrix through a metering pump.

45. The process of claim 43, wherein said step of degassing said matrix is performed after pumping said matrix through a metering pump.

46. The process of claim 43, further comprising the step of (d) degassing said active-containing degassed film-forming matrix.

47. The process of claim 43, further comprising the step of forming a wet film from said active-containing degassed film-forming matrix.

48. The process of claim 43, further comprising the step of determining whether additional degassing of said active-containing degassed film-forming matrix is required.

49. The process of claim 43, wherein said step of degassing is performed at a low pressure.

50. The process of claim 43, wherein said step of degassing is performed in a vacuum.

51. The process of claim 43, wherein said step of degassing is performed in-line.

52. The process of claim 43, wherein said step of degassing further comprises directing the matrix through a plurality of volume reduction devices in series.

53. The process of claim 43, wherein said step of degassing further comprises directing the matrix through a plurality of rotating plates in parallel.

54. A system for forming edible film, comprising:
(a) a first mixer for combining at least one self-supporting film forming matrix, including an edible polymer component and at least one fluid carrier;
(b) at least one volume reduction device for degassing said matrix; and
(c) at least one second mixer to combine a quantity of a degassed matrix with a quantity of an active component to form an active-containing degassed matrix having a uniform distribution of said active therein.

55. The system of claim 54, further comprising a metering pump for pumping a desired amount of matrix from said first mixer to said second mixer, and wherein said volume reduction device is disposed at a location between said first mixer and said metering pump.

56. The system of claim 54, further comprising a metering pump for pumping a desired amount of matrix from said first mixer to said second mixer, and wherein said volume reduction device is disposed at a location between said metering pump and said second mixer.

57. The system of claim 54, further comprising a second volume reduction device for degassing said active-containing degassed matrix.

58. The system of claim 54, further comprising a wet film forming device for forming a self-supporting edible film from said active-containing degassed matrix.

59. The system of claim 54, wherein said volume reduction device comprises at least one rotatable surface.

60. The system of claim 54, wherein said volume reduction device comprises a plurality of porous channels comprising a membrane, wherein said membrane is permeable to a gas and impermeable to a non-gaseous material.
